(19) United States
(12) Patent Application Publication
(10) Pub. No.: US 2026/0207362 A1
(43) Pub. Date: Jul. 23, 2026

(54) DELIVERY SYSTEMS AND METHODS

(71) Applicant: Boston Scientific Scimed, Inc., Maple Grove, MN (US)

(72) Inventors: Martin Burke, Loughrea (IE); Gerard Duignan, Loughrea (IE); Kevin McEvilly, Oranmore (IE); Louis McNern, Kinvara (IE); Orlaith Marie Duffy, Claremorris (IE); David Collins, Athenry (IE); Meredith Margaret Dixon, Galway (IE); Scott Stephen Lueders, Medfield, MA (US); Shawn Ryan, Littleton, MA (US)

(73) Assignee: Boston Scientific Scimed, Inc., Maple Grove, MN (US)

(21) Appl. No.: 19/455,099

(22) Filed: Jan. 21, 2026

Related U.S. Application Data
(60) Provisional application No. 63/748,798, filed on Jan. 23, 2025.

(51) Int. Cl. A61F 2/966; A61B 8/12; A61F 2/95; A61L 29/06; A61M 25/00

(52) U.S. Cl. CPC A61F 2/966; A61B 8/12; A61F 2/9517; A61L 29/06; A61M 25/0045; A61M 25/0053; A61M 25/0068

(57) ABSTRACT

Catheter assemblies, systems, and methods for delivering a stent or other tissue anchor to one or more vessel lumens are disclosed. A catheter assembly may include an outer sheath, an inner body, and a distal tip. The outer sheath may have an outer sheath lumen extending therethrough. The inner body may have a proximal end, a distal end, and an inner body lumen extending therethrough. A portion of the inner body may define a stent holding region. The distal tip may be positioned proximate to the distal end of the inner body. The stent holding region may be configured to hold a stent such that a distal end of the stent is spaced a distance proximal of a proximal end of the distal tip. The catheter assembly may include a flexible region between a distal end of the stent holding region and a proximal end of the distal tip.

### CROSS-REFERENCE TO RELATED APPLICATIONS

This application claims the benefit of U.S. Provisional Patent Application Ser. No. 63/748,798 filed on Jan. 23, 2025, the disclosure of which is incorporated herein by reference.

### FIELD

The present disclosure relates generally to medical methods and apparatus. More particularly, the present disclosure relates to methods, apparatus, and systems for delivering medical devices to a target location.

### BACKGROUND

A wide variety of intracorporeal and extracorporeal medical devices and systems have been developed for medical use, for example, for endoscopic procedures. Some of these devices and systems include guidewires, catheters, catheter systems, endoscopes, ablation devices, delivery systems, stents, anchors, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY OF THE DISCLOSURE

This disclosure provides design, material, manufacturing method, and use alternatives for medical devices, including stent delivery devices, systems, and methods.

In a first example, a catheter assembly may include an outer sheath having an outer sheath lumen extending therethrough, an inner body having a proximal end, a distal end, and an inner body lumen extending therethrough, a portion of the inner body defining a stent holding region, a distal tip positioned proximate to the distal end of the inner body, and a flexible region between a distal end of the stent holding region and a proximal end of the distal tip.

Alternatively or additionally to any of the examples above, the stent holding region may be configured to hold the stent such that the distal end of the stent is spaced from the proximal end of the distal tip a distance in a range of 3 millimeters (mm) to 8 mm.

Alternatively or additionally to any of the examples above, the distal tip may include a tapered tip having a proximal end having a first diameter, a distal end having a second diameter, and a guidewire lumen extending therethrough, wherein the first diameter is greater than the second diameter and a distal tip base disposed on the proximal end of the tapered tip, the guidewire lumen may extend through the distal tip base, and wherein the tapered tip may have a first length and the distal tip base has a second length that is shorter than the first length.

Alternatively or additionally to any of the examples above, the distal tip base may have a length of 1 mm.

Alternatively or additionally to any of the examples above, the distal tip may be more rigid than the inner body at a portion of the inner body between the distal end of the stent holding region and the proximal end of the distal tip.

Alternatively or additionally to any of the examples above, the inner body may have a plurality of layers including an inner layer formed of a polyimide, an outer layer formed of a polyether block amide, and a middle layer formed of a braided material.

Alternatively or additionally to any of the examples above, the inner body may include a braided layer having a first per-inch-cross (PIC) count distal of a distal end of the stent holding region and a second PIC count proximal of a proximal end of the stent holding region, the first PIC count is greater than the second PIC count.

Alternatively or additionally to any of the examples above, the inner body may include a braided layer having first pattern distal of a distal end of the stent holding region and a second pattern proximal of a proximal end of the stent holding region.

Alternatively or additionally to any of the examples above, the inner body may include a braided layer formed from a wire having a first diameter distal of a distal end of the stent holding region and a second diameter proximal of a proximal end of the stent holding region, the second diameter is greater than the first diameter.

Alternatively or additionally to any of the examples above, the inner body may be formed from one or more polymer materials having a durometer in a range of 40 Shore D to 90 Shore D.

Alternatively or additionally to any of the examples above, the catheter assembly may include a handle coupled with the inner body and the outer sheath, wherein actuation of a first portion of the handle may advance the inner body in a first direction relative to the outer sheath and actuation of a second portion of the handle may advance the outer sheath relative to the inner body and in a second direction opposing the first direction.

In a further example, a catheter assembly may include an outer sheath having an outer sheath lumen extending therethrough, an inner body having a proximal end, a distal end, and an inner sheath lumen extending therethrough, a portion of the inner body receivable within the outer sheath lumen of the outer sheath, a distal tip positioned proximate to the distal end of the inner body and the distal tip including a tapered tip having a proximal end having a first diameter, a distal end having a second diameter, and a guidewire lumen extending therethrough, wherein the first diameter is greater than the second diameter, a distal tip base disposed on the proximal end of the tapered tip, the guidewire lumen extending through the distal tip base, and a conductive cutting member coupled with the tapered tip, and a stent constrained on the inner body with a distal end of the stent spaced from a proximal end of the distal tip base.

Alternatively or additionally to any of the examples above, the catheter assembly may further include a stent holder coupled with the inner body and configured to couple the stent relative to the inner body with the distal end of the stent spaced from the proximal end of the distal tip base.

Alternatively or additionally to any of the examples above, a distance between the distal end of the stent and the proximal end of the distal tip base may be in a range of 3 millimeters (mm) to 8 mm.

Alternatively or additionally to any of the examples above, the tapered tip may have a first length and the distal tip base may have a second length that is shorter than the first length.

Alternatively or additionally to any of the examples above, the distal tip may be more rigid than the inner body at a portion of the inner body between the distal end of the stent and the proximal end of the distal tip base.

Alternatively or additionally to any of the examples above, the inner body may be formed from one or more polymer materials having a durometer in a range of 40 Shore D to 90 Shore D.

In a further example, a method of using a catheter assembly may include engaging a distal end of an endoscope having endoscopic ultrasound (EUS) visualization with a surface of or in contact body tissue defining a first body lumen, advancing the catheter assembly through the endoscope, while a stent is constrained between the inner body and an outer sheath of the catheter assembly, inserting a guidewire through the endoscope and into a second body lumen, advancing an inner body and a distal tip of the catheter assembly into the second body lumen over the guidewire, advancing the inner body over the guidewire and relative to the outer sheath in a first direction to deploy a distal end of the stent within the second body lumen, and advancing the outer sheath relative to the inner body in a second direction to deploy a proximal end of the stent within the first body lumen.

Alternatively or additionally to any of the examples above, the distal end of the endoscope may be engaged with the surface of the body tissue defining the first body lumen with the distal tip and the inner body in the second body lumen and while advancing the inner body relative to the outer sheath to deploy the distal end of the stent within the second body lumen.

Alternatively or additionally to any of the examples above, the distal end of the stent may be spaced proximal of a proximal end of the distal tip while the stent is constrained between the inner body and the outer sheath.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative configuration of a catheter assembly;

FIG. 2 is a schematic partial cross-section view and partial side view of a distal portion of an illustrative configuration of a catheter assembly, with an outer sheath in cross-section and all other components in a side view;

FIG. 3 is a schematic partial cross-section view and partial side view of a distal portion of an illustrative configuration of a catheter assembly, with an outer sheath in cross-section and all other components in a side view;

FIG. 4 is a schematic cross-section view of a distal portion of an illustrative configuration of a catheter assembly;

FIG. 5 is a schematic box diagram of an illustrative method of using a catheter assembly; and

FIGS. 6A-6E schematically depicts steps in a technique for delivering a stent to a target location and deploying the stent at the target location.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative configurations and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

A number of inter and intra-luminal endoscopic procedures require precise placement of anchors or stents. For example, a number of procedures may be performed by entering the gastrointestinal (GI) tract through a first organ or structure, such as the esophagus, stomach, duodenum, small intestine, or large intestine, and delivering the anchor or stent to adjacent organs and lumens or tissue structures such as an adjacent portion of the GI tract, the bile duct, the pancreatic duct, the gallbladder, the pancreas, cysts, pseudocysts, abscesses, and the like. While primarily intended for use in the GI tract, such methods and apparatus can also be used for access to and from portions of the urinary tract, such as the urinary bladder and ureter, the pulmonary tract, such as the trachea and bronchi, and the biliary tract, such as the bile duct and gallbladder, as well.

Intra-ductal stents are commonly used to facilitate the opening of closed vessels for access, drainage or other purposes. Tissue anchors are used to secure adjacent tissues or organs. Inter-luminal tissue anchors, which include a central lumen, are used to facilitate fluid communication between adjacent ducts, organs, or lumens. The precise placement of the tissue anchor or stent is necessary, especially when the tissue anchor or stent has well defined anchoring elements at the proximal and/or distal ends, and the device is used to secure adjacent lumens.

When deploying a stent or other tissue anchor between adjacent body lumens, organs, or other structures, it is typically necessary to penetrate both a wall of the first body lumen through which access is established and a wall of a second body lumen which is the target for the procedure. When the second body lumen has a diameter that is small, an angle of access into the second vessel may not allow the catheter delivering the stent or other tissue anchor to travel deep into the second body lumen. Due to the limited travel depth into the second body lumen, it may be difficult to maintain a correct position of the distal end of the catheter during deployment of the stent or other tissue anchor to ensure the stent or other tissue anchor is not mis-deployed (e.g., such that that the stent or other tissue anchor is deployed at a desired location across the second body lumen and the first body lumen).

When stents (e.g., braided stents, lumen-apposing metal stents (LAMS), etc.) are deployed, the stents may foreshorten significantly which may complicate deploying stents coupling the first body lumen with the second body lumen. A method of deploying a stent that is likely to foreshorten as the stent expands may include advancing in a proximal direction an outer sheath covering or radially constraining the stent on an inner body to allow a distal end of the stent to expand (e.g., self-expand) in a controlled manner. As the outer sheath is advanced proximally to deploy the stent, the stent may foreshorten as distal end (e.g., a distal flange) of the stent is exposed and expands. The foreshortening of the stent may result in the distal end of the stent deploying outside of or proximal of the second body lumen or other suitable target site.

Techniques have been developed to address foreshortening of a stent and related complications of placing a stent or other tissue anchor. In one example, a deployment mechanism of a handle of a stent or anchor delivery system may require a movement that advances the stent and an inner tube supporting the stent in a distal direction relative to the outer sheath and into the second body lumen to deploy the stent within the second body lumen. A benefit of this technique is that as the inner tube pushes forward, the forward movement of the stent counteracts foreshortening of the stent as the stent expands such that the distal end of the stent is deployed at the target site (e.g., within the second body lumen) and mitigates a likelihood of mis-deployment of the distal end of the stent. A limitation of this technique, however, is that a tip of the delivery system can only advance forward so far without engaging a far side of tissue defining the second body lumen, which may oppose a near side of the tissue defining the second body lumen through which the distal end of the delivery system enters the second body lumen.

During stent deployment, endoscope ultrasonic (EUS) visualization may be utilized to visualize a target site during for the stent. When EUS visualization is utilized, a distal end of the endoscope is maintained 1) in contact with tissue defining the first body lumen and that is in contact with the tissue defining second body lumen or 2) in contact with the tissue defining the second body lumen. However, if the inner tube is advanced into the second body lumen to deploy the stent and the distal tip engages the far side of tissue defining the second body lumen, the tissue defining the second body lumen may be moved away from the distal end of the scope such that visualization of the second body lumen may be compromised.

Improved catheter designs are disclosed herein. The designs disclosed herein offer a number of advantages over prior art designs. The devices disclosed herein may be configured to deliver and deploy a stent or other tissue anchor at a precise target site, while mitigating risk associated with foreshortening of the stent or other tissue anchor and/or engaging tissue defining the second body lumen. The devices disclosed herein may prevent or mitigate loss of visualization as the stent, the inner tube, and/or the outer sheath advance into the second body lumen.

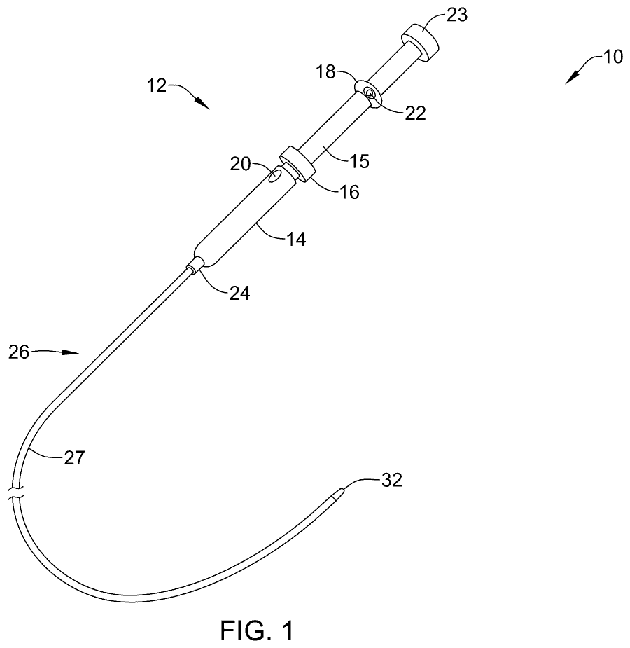

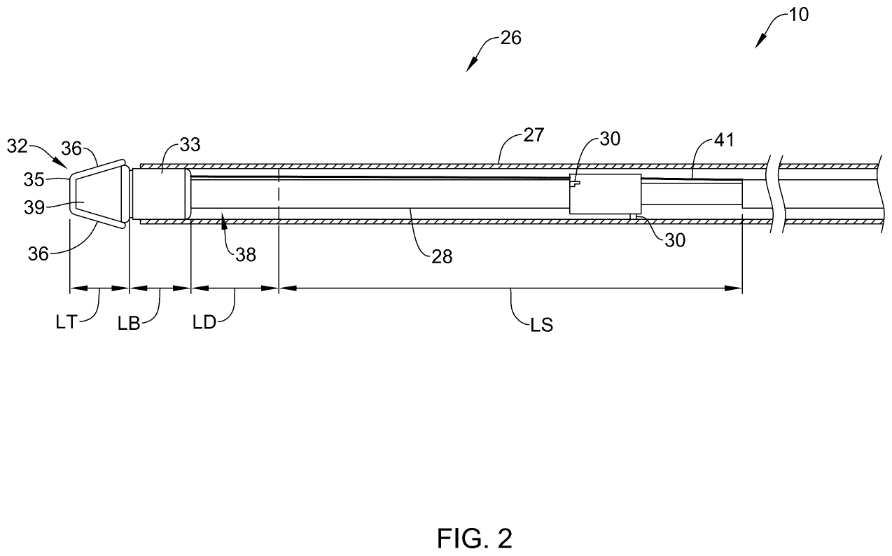

Turning to the Figures, the device 10 (e.g., a catheter assembly) of FIG. 1 may include a control handle 12 having a body 14 with a first slide actuator 15 with a knob 16 and a lock 20. Further, the device 10 may include a second slide actuator 18 with a lock 22, a scope locking mechanism 24 (e.g., a scope lock), an electrical plug 23, a catheter body 26, an outer sheath 27, and a distal tip 32. The handle 12 may be coupled with the outer sheath 27 and an inner body (e.g., an inner body 28 discussed below with respect to FIG. 2). Actuation of a first portion (e.g., the first slide actuator 15) of the handle 12 may advance the inner body in a first direction relative to the outer sheath 27 and actuation of a second portion (e.g., the second slide actuator 18) of the handle 12 may advance the outer sheath 27 in a second direction opposing the first direction.

FIG. 2 schematically depicts a view of a portion of a distal region of the catheter body 26 of the device 10, where the outer sheath 27 is depicted in a cross-section view and the remaining portions of the device 10 are depicted in a side view. In some examples, the distal region of the catheter body 26 depicted in FIG. 2 may be configured to allow the distal region to travel into the second body lumen during deployment of a distal end of a stent or other tissue anchor while minimizing or mitigating forces imparted on a wall defining the second body lumen and thus maintaining a position of a scope relative to the tissue defining the second body lumen during deployment of the distal end of the stent or other tissue anchor (e.g., deployment of a first flange of the stent or other tissue anchor).

As depicted in FIG. 2, the distal region of the catheter body 26 may include the outer sheath 27 having a lumen (e.g., an outer sheath lumen extending therethrough), an inner body 28 configured to be received within the lumen of the outer sheath 27, the distal tip 32 at or proximate the distal end of the inner body 28, one or more stent locks 30, and/or other suitable components. In some examples, a cutting element 35 may be located at the distal tip 32 (e.g., coupled to a tapered portion 39 of the distal tip 32) and one or more conductive projections 36 may extend from the cutting element 35. The cutting element 35 may be, or may be electrically coupled with, an electrode 37, which may be coupled with an electrical power source (e.g., via the handle 12). Example suitable cutting elements 35 and/or other components discussed herein include, but are not limited to, example cutting elements and/or other components disclosed in U.S. application Ser. No. 17/820,489, filed on Aug. 17, 2022, titled METHODS AND DEVICES FOR ACCESS ACROSS ADJACENT TISSUE LAYERS, which is hereby incorporated by reference in its entirety for any and all purposes.

The distal tip 32 may include a conductive portion with a cutting element 35. The illustrated cutting element 35 may have a concentric design about a guidewire lumen 48 and/or other suitable configuration. The conductive projections 36 may extend from the cutting element 35 towards the outer diameter of the distal tip 32. The illustrated projections 36 may enter into a recessed portion of the distal tip 32 and/or other suitable portion of the distal tip 32.

The conductive areas of the distal tip 32, such as the cutting element 35 and the projections 36 may be configured to cut, heat, and/or cauterize tissue in a patient. Electrical energy may be supplied to energize the conductive portions of the distal tip 32 via one or more electrodes 41. The electrical energy may be supplied through the electrical plug 23, which may be electrically coupled with the electrode 41. The handle 12 may include an electrical control to control the electrical energy supplied to the distal tip 32. Any suitable electrical energy may be supplied to the conductive portions of the distal tip 32 including, but not limited to, radiofrequency (RF), high-frequency (HF) energy, and/or other suitable electrical energy.

The cutting element 35 and illustrated projections 36 can be made out of any suitable material(s). In some examples, the cutting element 35 and the illustrated projections 36 may be made from a conductive medical grade material that is biocompatible, such as stainless steel. A different conductive material, such as copper, may be used for the electrode 37 to supply electrical energy to the cutting element 35 and projections 36. Other suitable materials electrically conductive materials may be utilized.

The distal tip 32 may have any suitable configuration. In some examples, the distal tip 32 may include a base portion 33 (e.g., a distal tip base) and a tapered portion 39 (e.g., a tapered tip) extending in a distal direction from the base portion 33. In some examples, the outer sheath 27 may contact the distal tip 32 and engage with a surface at an outer diameter of the base portion 33 of the distal tip 32.

The tapered portion 39 may have a proximal end having a first diameter, a distal end having a second diameter, where the first diameter may be greater than the second diameter. The base portion 33 may be disposed at or on the proximal end of the tapered portion 39 and have an outer diameter equal to or less than the proximal end of the tapered portion 39. The guidewire lumen 48 may extend through the inner body 28, along with the base portion 33 and the tapered portion 39 of the distal tip 32.

The distal tip 32 may have any suitable size. In some examples, the distal tip 32 may have a length in a range of about 1 millimeter (mm) to about 20 mm, in a range of about 2 mm to about 10 mm, and/or in one or more other suitable ranges. In some examples, the base portion 33 and the tapered portion 39 of the distal tip 32 may have a same or similar length or may have different lengths. In some examples, the distal tip 32 may have a length of less than 8 mm. In one example configuration of the distal tip 32, the distal tip 32 may have a length of 6 mm. In one example configuration of the distal tip 32, the distal tip 32 may have a length of 4 mm.

The base portion 33 of the distal tip 32 may have any suitable length L_{B} (e.g., a second tip length) and the tapered portion 39 of the distal tip 32 may have any suitable length L_{T} (e.g., a first tip length). In some examples, the length L_{B} of the base portion 33 may be less than, equal to, or greater than the length L_{T} of the tapered portion 39. In some examples, the length L_{B} of the base portion 33 may have a length in a range of 1 mm to about 5 mm. In some examples, the length L_{T} of the tapered portion 39 may have a length in a range of 1 mm to about 5 mm. In one example, the length L_{B} of the base portion 33 may be about 3 mm and the length L_{T} of the tapered portion 39 may be about 3 mm. In one example, the length L_{B} of the base portion 33 may be about 1 mm and the length L_{T} of the tapered portion 39 may be about 3 mm. In some examples, the base portion 33 of the distal tip 32 may be omitted. Other suitable configurations of the distal tip 32 are contemplated. In some examples, having a short distal tip 32, a short base portion 33, and/or omitting the base portion 33 may facilitate having a flexible region 38 of the device 10 that may be flexible and/or configured to bend between the distal tip 32 and a distal end of the stent or other tissue anchor to facilitate inserting the distal end of the stent or other tissue anchor to a desired location in the second body lumen without moving tissue defining the second body lumen away from a visualization component. Such a configuration of the distal tip 32 may allow the flexible region 38 to deflect with less force than when the distal tip 32 and/or the base portion 33 are longer.

The distal tip 32 may be formed from any suitable material. In some examples, the distal tip 32 may be made out of an insulating material to insulate the cutting element 35 and projections 36 from the surrounding device structure. In some examples, the distal tip 32 may be formed from a material that is more rigid than material forming the inner body 28. For example, the distal tip 32 may be more rigid than the inner body 28 at a portion of the inner body between the distal end of the stent (e.g., a distal end of a stent holding region) and a proximal end of the distal tip 32 (e.g., at the flexible region 38). In one example, the distal tip 32 may be formed from a ceramic material, but other suitable materials may be utilized for the distal tip 32.

The outer sheath 27 may be configured to longitudinally and/or radially constrain the stent or other tissue anchor and prevent the stent or other tissue anchor from expanding and/or foreshortening. As such, as the outer sheath 27 is advanced in a proximal direction (e.g., withdrawn) relative to the stent or other tissue anchor and/or as the inner body 28 is advanced with the stent or other tissue anchor in a distal direction relative to the outer sheath 27, the stent or other tissue anchor that is no longer covered by the outer sheath 27 may begin to deploy and/or otherwise expand and, possibly, foreshorten. In some examples, when the stent or other tissue anchor is covered by the outer sheath 27, the stent or other tissue anchor may have a length L_{S}.

The stent lock(s) 30 (e.g., stent holder(s)) may be coupled with the inner body 28 and configured to engage or couple with the stent or other tissue anchor to maintain the stent or other tissue anchor at a longitudinal location along the inner body 28 (e.g., at a stent holding region) when the outer sheath 27 is covering the stent or other tissue anchor. The stent lock(s) may have any suitable configuration configured to engage the stent or other tissue anchor. In some examples, the stent lock(s) 30 may have a wing configuration, a hook configuration, a protrusion configuration, and/or other suitable type of configuration designed to maintain a longitudinal position of the stent or other tissue anchor when the stent or other tissue anchor is covered by the outer sheath 27. In one example and as depicted in FIG. 2, the stent locks 30 may have a wing and/or hook configuration, but other suitable configurations are contemplated.

Any suitable number of stent locks 30 may be utilized to engage the stent and the stent locks 30 may be at any suitable location relative to one another. In some example, a single stent lock 30 or two or more stent locks 30 may be utilized. In some examples, when two or more stent locks 30 are utilized, the stent locks 30 may be longitudinally and/or circumferentially spaced from one another.

The stent lock(s) 30 may be located at any suitable location along the inner body 28. In some examples, the stent lock(s) 30 may be located along the inner body 28 at any suitable location configured to maintain a distal end of the stent or other tissue anchor a desired distance L_{D} proximal of a proximal-most end of the distal tip 32 (e.g., a proximal-most end of the base portion 33, when included).

The length or distance L_{D} between the distal tip 32 and the stent or other tissue anchor may be any suitable length or distance. In some examples, the length or distance L_{D} may be configured to facilitate creating the flexible region 38 of the device 10 such that the flexible region 38 is flexible and/or may be configured to bend between the distal tip 32 and a distal end of the stent or other tissue anchor to facilitate inserting the distal end of the stent or other tissue anchor to a desired location in the second body lumen without moving tissue defining the second body lumen away from a visualization component. In some examples, the length or distance L_{D} may have a length in a range of 1 mm to 20 mm, a range of 1 mm to 10 mm, a range of 1 mm to 8 mm, a range of 3 mm to 8 mm, and/or other suitable range. In one example, the length or distance L_{D} may be 8 mm. Other suitable lengths or distances L_{D} are contemplated.

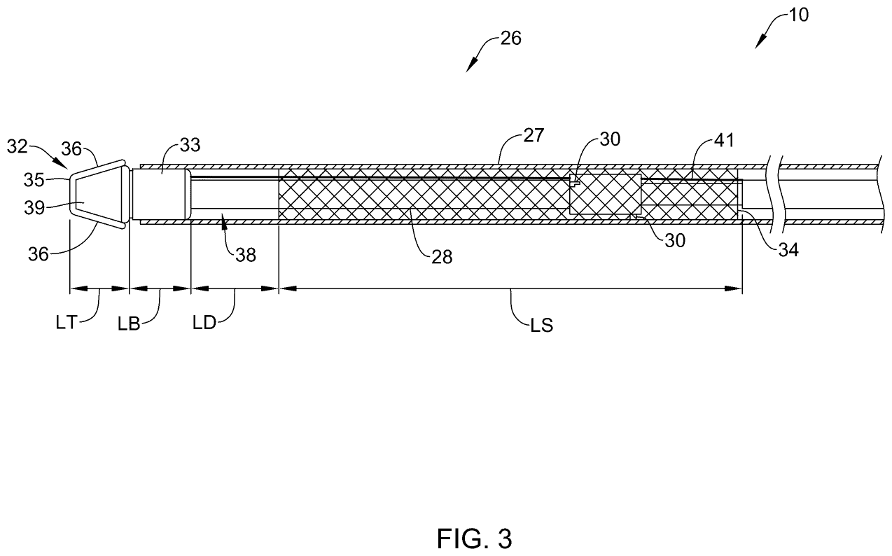

FIG. 3 depicts a schematic view of a portion of a distal region of the catheter body 26 of the device 10, where the outer sheath 27 is depicted in a cross-section view and the remaining portions of the device 10 are depicted in a side view. The portion of the distal region of the catheter body 26 depicted in FIG. 3 may be similar to or the same as that depicted in FIG. 2, but with a stent 34 coupled with the stent locks 30 and constrained between the inner body 28 and the outer sheath 27 by the outer sheath 27.

As depicted in FIG. 3, the constrained stent 34 may be coupled with the stent locks 30 such that the flexible region 38 may have a length or distance L_{D} configured to facilitate inserting the distal end of the stent 34 within the second body lumen without pushing the tissue defining the second body lumen away from a visualization tool. For example, the flexible region 38 may facilitate the distal region of the catheter body tracking a guidewire inserted through the catheter body 26 and into the second body lumen to facilitate deploying the stent 34 at a desired location within the second body lumen. Although not depicted in FIG. 3, the outer sheath 27 may be configured to extend to the distal end of the stent 34, but not to a proximal end of the distal tip 32 to facilitate reducing a stiffness of the catheter body 26 along the flexible region 38 and allow the flexible region to bend or flex as the catheter body 26 traverses a guidewire and is positioned within the second body lumen. Such a positioning of the outer sheath 27 may allow the flexible region 38 to deflect with less force than when the outer sheath 27 extends to the distal tip 32 and/or the base portion 33.

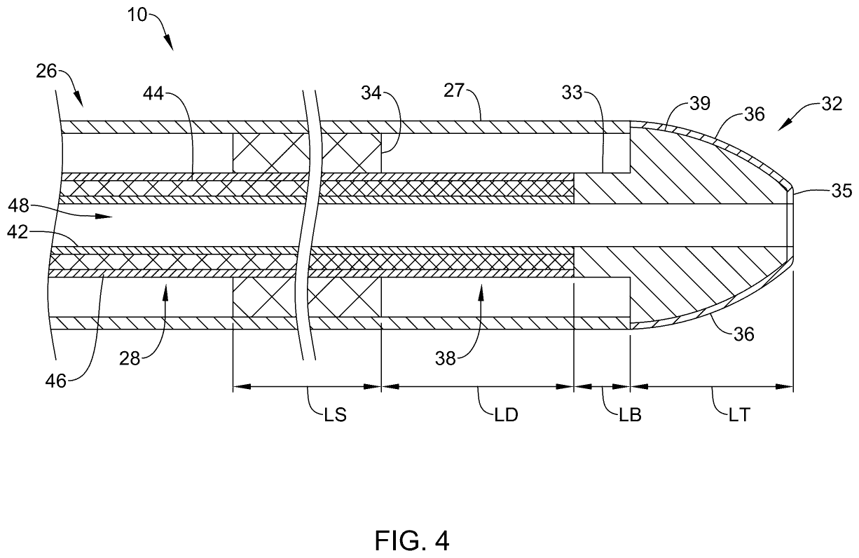

FIG. 4 depicts a schematic cross-section view of a portion of a distal region of the catheter body 26 of the device 10. As depicted in FIG. 4, the outer sheath 27 may define a lumen configured to receive the inner body 28 and the stent 34 and may constrain the stent 34.

The inner body 28 may have any suitable configuration. In some examples, the inner body 28 may have a proximal end (not shown in FIG. 4), a distal end, and an inner body lumen. In some examples, the inner body lumen may be or may be part of the guidewire lumen 48 and may be configured to receive a guidewire. The inner body lumen may extend from the proximal end to the distal end of the inner body 28, but other suitable configurations are contemplated where the inner body lumen does not extend through one or both of the proximal end and the distal end of the inner body 28.

A portion of the inner body 28 between the proximal end and the distal end of the inner body 28 may define a stent holding region. In some examples, the stent holding region may extend the length or distance L_{S} and/or may be located along the inner body 28 at the length or distance L_{S}, but other suitable configurations are contemplated. The stent holding region may be configured to hold the stent 34 such that a distal end of the stent is spaced in a proximal direction from a proximal end of the distal tip a length or distance L_{D}.

The inner body 28 may have one or more layers of materials. As depicted in FIG. 4 for example, the inner body 28 may include an inner layer 42 defining the guidewire lumen 48 extending through at least a portion of the catheter body 26, a middle layer 44, and an outer layer 46. Other suitable layers may be utilized to form the inner body 28.

The inner body 28 may be formed from one or more materials. In some examples and as depicted in FIG. 4, the inner body 28 may be formed from a plurality of materials. In some examples, a portion of the inner body 28 at the flexible region 38 may be configured to facilitate flexing and/or bending of the inner body 28 at the flexible region 38. The layers of the inner body 28 may be formed from any suitable material(s) including, but not limited to, polymers, metals, stainless steel, polymide, polyether block amides (e.g., PEBAX, etc.), and/or other suitable materials. The materials of the layers may have any suitable configuration including, but not limited to, a tube, a coating, a braid, and/or other suitable configurations. In one example configuration, the inner body 28 may be formed from an inner layer 42 of a polyimide, a middle layer 44 of a braided material, and an outer layer of a polyether block amide. In one example configuration, the inner body 28 may be formed from an inner layer 42 of a polyimide tube, a middle layer 44 of a stainless steel braided tube, and an outer layer of a polyether block amide tube. When a polymer material is used for a layer of the inner body 28, the polymer material may have a durometer in a range of 40 Shore D to 90 Shore D, in a range of 50 Shore D to 90 Shore D, or other suitable durometer. Other suitable material configurations are contemplated.

In some examples, materials and/or configurations of materials may be utilized to facilitate allowing the flexible region 38 to bend or flex as the catheter body 26 traverses a guidewire into the second body lumen or other suitable body lumen. Such materials and/or configurations of materials at the flexible region 38 may allow the flexible region 38 to deflect with less force than when other materials or configurations of materials are used at the flexible region 38. In some examples, a portion of the inner body 28 at the flexible region 38 may have a configuration designed to create a more flexible inner body 28 than at one or more locations proximal of the flexible region 38. Example configurations of the layers of the inner body 28 at the flexible region 38 may include, but are not limited to, an adjustment of a braid pattern of the middle layer 44 at the flexible region 38 relative to a location proximal of the flexible region 38, an increased per inch crosses (PIC) count of the braid of the middle layer 44 at the flexible region 38 relative to a location proximal of the flexible region 38 (e.g., as depicted in FIG. 4), reducing a wire diameter of the braid of the middle layer 44 at the flexible region 38 relative to a location proximal of the flexible region, changing the polymer material or non-polymer material of the inner layer 42, the middle layer 44, and/or the outer layer 46 at the flexible region 38 relative to a location proximal of the flexible region 38, and/or the flexible region 38 may have other suitable configurations and/or materials that facilitate the flexible region 38 of the inner body 28 being more flexible than other regions proximal of the flexible region 38. In some examples, the materials and/or configurations of the layers at the flexible region 38 may extend some distance proximal of the flexible region 38, but other suitable configurations are contemplated. In one example configuration and as depicted in FIG. 4, the inner body 28 at the flexible region 38 may include a braided middle layer 44 having a greater PIC count at the flexible region 38 than at locations proximal of the stent 34. In one example configuration, the inner body 28 may include a braided layer (e.g., the braided layer may be the middle layer 44 and/or one or more other layer) having a first PIC count distal of a distal end of the stent holding region and a second PIC count proximal of a proximal end of the stent holding region, where the first PIC count may be greater than the second PIC count, as depicted for example in FIG. 4. In one example configuration, the inner body 28 may include a braided layer (e.g., the braided layer may be the middle layer 44 and/or one or more other layer) having a first pattern distal of a distal end of the stent holding region and a second pattern proximal of a proximal end of the stent holding region. In one example configuration, the inner body 28 may include a braided layer (e.g., the braided layer may be the middle layer 44 and/or one or more other layer) formed from a wire having a first diameter distal of a distal end of the stent holding region and a second diameter proximal of a proximal end of the stent holding region, where the second diameter may be greater than the first diameter. Other suitable configurations are contemplated.

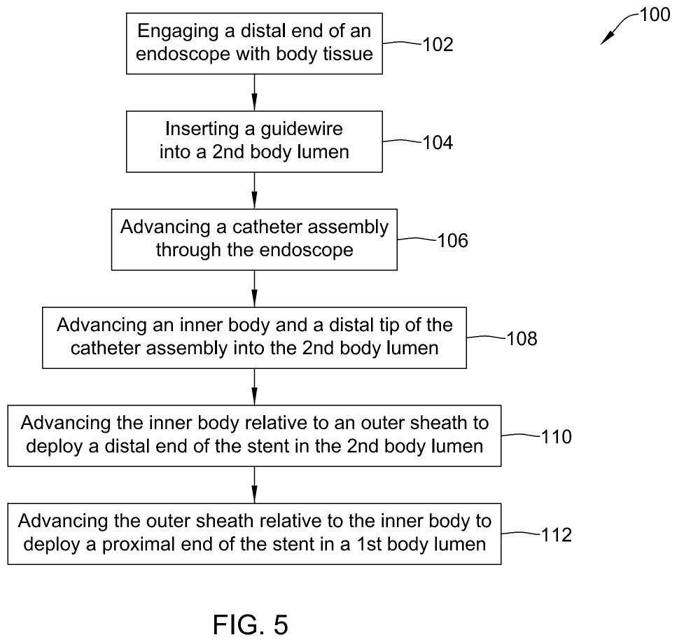

FIG. 5 depicts a schematic box diagram of an illustrative method 100 of using a catheter assembly. The devices discussed herein and/or other suitable devices may be utilized to effect the method 100. In some examples, the method 100 may be utilized to place a stent or other tissue anchor between a first body lumen and a second body lumen to fluidly couple the first body lumen with the second body lumen.

The method 100 may include engaging 102 a distal end of an endoscope with body tissue defining the first body lumen. In some examples, the endoscope may include endoscope ultrasound (EUS) visualization. To best visualize the first body lumen and/or the second body lumen during the method 100, the distal end of the endoscope or other portion of the endoscope may be in contact with tissue defining the first body lumen and contacting tissue defining the second body lumen. Other suitable types of visualization may be utilized, which may require other suitable positioning of the endoscope.

The catheter assembly may be advanced 104 through the endoscope (e.g., through the working channel of the endoscope) to the body tissue defining the first body lumen. In some examples, the catheter assembly may be advanced through a working channel of the endoscope and/or through one or more other suitable channels of the endoscope. In some examples, the catheter assembly may include a stent constrained on an inner body by an outer sheath as the catheter assembly is advanced through the endoscope to the body tissue defining the first body lumen.

Once a distal tip of the catheter assembly is at the body tissue defining the first body lumen, a cutting element of the catheter assembly may be utilized to create an opening through the body tissue defining the first body lumen and the body tissue defining the second body lumen. In some examples, the cutting element may be electrically conductive and electrical energy may be applied to the cutting element to facilitate creating the opening through the body tissue defining the first body lumen and the body tissue defining the second body lumen. As an alternative to or in addition to utilizing the cutting element of the catheter assembly, a needle may be used to create an initial opening through the body tissues defining the first body lumen and the second body lumen.

The method 100 may include inserting 106 a guidewire into the second body lumen. In some examples, the guidewire may be passed into to the first body lumen through the working channel of the endoscope and/or through a guidewire lumen of the catheter assembly and through the opening of the tissues defining the first body lumen and the second body lumen.

With the guidewire in the second body lumen, an inner body and a distal tip of the catheter assembly may be advanced 108 into the second body lumen. In some examples, a portion of the inner body distal of the constrained stent may be advanced into the second body lumen over the guidewire. The distal tip and a flexible portion of the inner body distal of a distal end of the stent may be configured to traverse the guidewire inserted in the second body lumen without engaging tissue defining the second body lumen or engaging tissue defining the second body lumen opposite a side of the tissue through which an opening was created with the cutting element with insufficient force to move the tissue defining the second body lumen. As the body tissue defining the second body lumen does not move as the catheter assembly advances over the guidewire into the second body lumen, sufficient visualization of the second body lumen may be achieved to perform the procedure of placing the stent such that the stent extends between the first body lumen and the second body lumen.

After the distal tip and the flexible region of the inner body are positioned in the second body lumen, the catheter assembly may be positioned such that a distal end of the stent and outer sheath are in or at the second body lumen. When so positioned, the inner body and/or the distal tip of the catheter assembly may be advanced 110 in a distal direction relative to the outer sheath to deploy the distal end of the stent (e.g., a distal flange of the stent) in the second body lumen. In some examples, the stent may advance in the distal direction with the inner body relative to the outer sheath. The stent may be a self-expanding stent and/or the stent may be expanded in one or more other manners. Further, the endoscope may remain engaged with a surface of or in contact with body tissue defining the first body lumen while the inner body and/or the distal tip are advanced relative to the outer sheath to deploy the distal end of the stent within the second body lumen. In some examples, advancing 108 the inner body and the distal tip of the catheter assembly into the second body lumen may be omitted or may be included as part of the advancing 110 the inner body and/or the distal tip of the catheter in a distal direction relative to the other sheath to deploy the distal end of the stent in the second body lumen.

Once the distal end of the stent has been deployed, the outer sheath may be advanced 112 in a proximal direction relative to the inner body to deploy a proximal end of the stent in the first body lumen. As the outer sheath is withdrawn, the stent (e.g., a proximal flange of the stent) may expand within the first body lumen to couple the first body lumen with the second body lumen.

After the stent has been deployed (e.g., expanded), the guidewire and catheter assembly may be withdrawn from the first body lumen and the second body lumen with or without the endoscope. The method 100, as discussed herein, may facilitate deploying the stent in the first body lumen and the second body lumen while utilizing EUS visualization to view the body lumens and procedure and while allowing a physician to account for foreshortening of a stent during placement and/or deployment.

Although certain steps of the method 100 were discussed herein, additional or alternative steps may be utilized. Further, although some steps of the method 100 were discussed temporally, the steps may be performed in one or more other suitable orders.

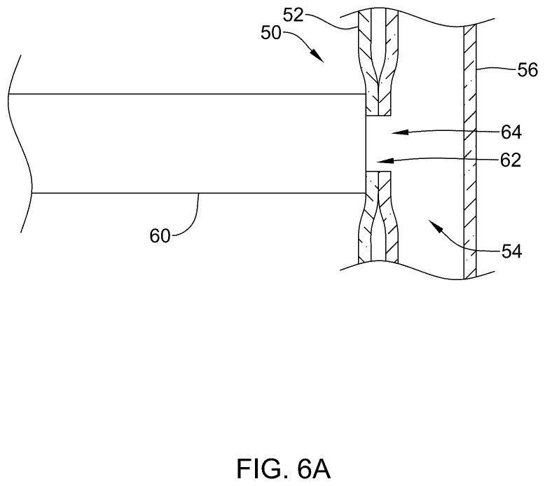

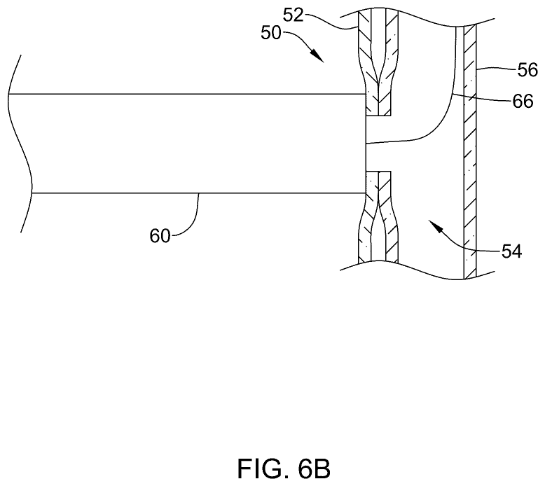

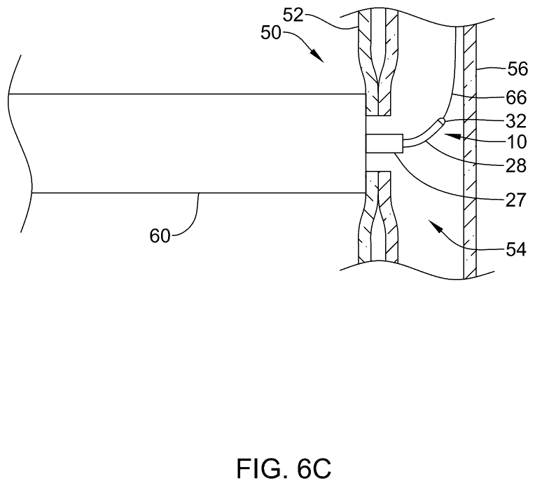

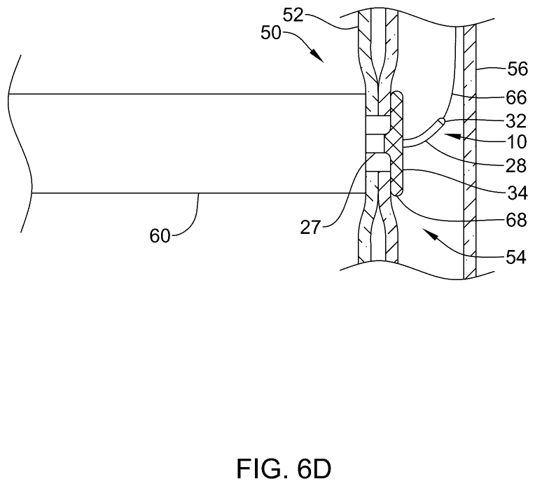

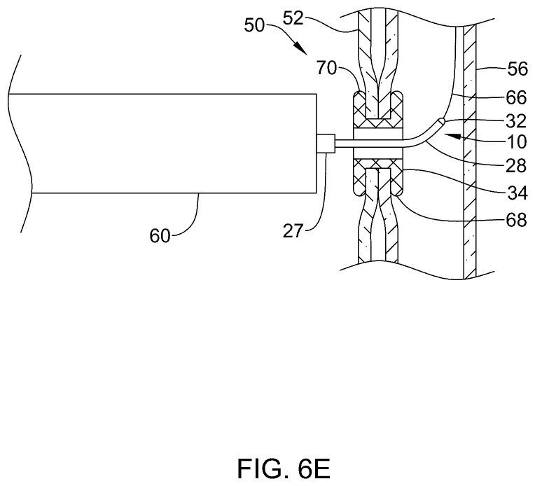

FIGS. 6A-6E depict an illustrative example of placing a stent in a first body lumen 50 defined by a first body tissue 52 and a second body lumen 54 defined by a second body tissue 56. In some examples, the device 10 may be attached to or extend through an endoscope 60 (e.g., an EUS endoscope). The distal end of the endoscope 60 (FIG. 6A-6E) may be positioned via trans-oral entry at or proximate to a target location within the gastrointestinal (GI) tract, however, the endoscope 60 may be placed at or proximate to a target location via one or more other access points.

Initial access between the first body lumen 50 and the second body lumen 54 may be effected using a needle (e.g., a 19 gauge needle, an electrosurgical needle, and/or other suitable needle). For example, a puncture may be made at the desired location using the needle followed by placement of a guidewire through the needle lumen. In some examples, a guidewire may be omitted. In addition to or as an alternative to using the needle, the device 10 may be used for creating initial access between the first body lumen 50 and the second body lumen 54. When the device 10 is used, electrical energy may be provided to the cutting element 35 and projections 36 to make an initial puncture in the first body tissue 52 and the second body tissue 56 to create the access between the first body lumen 50 and the second body lumen 54.

As depicted in FIG. 6A, a distal end of the endoscope 60 may be engaged with the first body tissue 52, which may contact the second body tissue 56, proximate the openings 62, 64 through the first body tissue 52 and the second body tissue 56. When the endoscope 60 is so positioned, the procedure for positioning a stent in communication with the first body lumen 50 and second body lumen 54 may be visualized using ultrasound visualization capabilities of the endoscope 60. Although not depicted in FIG. 6A, the catheter body 26 may be advanced over a guidewire through the working channel of the endoscope 60 and/or a guidewire lumen of the device 10 and positioned proximate the distal end of the endoscope 60.

As depicted in FIG. 6B, the guidewire 66 may be advanced through the guidewire lumen of the device 10, through the opening 62 in the first body tissue 52, through the opening 64 in the second body tissue 56, and into the second body lumen 54. The guidewire 66 may be advanced to a position in the second body lumen 54 such that the distal tip 32 and the inner body 28 may track over the guidewire 66 into the second body lumen 54 without engaging a wall of the second body tissue 56 or engaging the wall of the second body tissue 56 with an amount of force less than an amount force required to cause the second body tissue 56 proximate the opening 64 therethrough from separating from the first body tissue 52.

As depicted in FIG. 6C, the handle 12 (not depicted in FIG. 6C) of the device 10 may be adjusted to move the assembly of catheter body 26 including the outer sheath 27 and the inner body 28 having the stent 34 (not depicted in FIG. 6C) constrained therebetween in the distal direction and through the first opening 62 in the first body tissue 52 and the second opening 64 in the second body tissue 56. Further, the distal tip 32 and the inner body 28 may be advanced over the guidewire and into the second body lumen 54, as depicted in FIG. 6C.

As depicted in FIG. 6D, the handle 12 (not depicted in FIG. 6D) may be adjusted to move the distal tip 32 and the inner body 28 relative to the outer sheath 27 and in a distal direction over the guidewire 66. Advancing the inner body 28 in a distal direction relative to the outer sheath 27 may cause a distal end of stent 34 to extend into the second body lumen 54 distal of a distal end of the outer sheath 27 and a first flange 68 at the distal end of the stent 34 may expand at a desired location within the second body lumen 54. Advancing the inner body 28 into the second body lumen 54 may facilitate deploying the first flange 68 of the stent 34 at the desired location even though the stent 34 may foreshorten as the stent 34 expands. Due to the flexible region 38 of the inner body 28 (e.g., along the catheter body 26), the distal tip 32 and the inner body 28 may track over the guidewire 66 without engaging a wall of the second body tissue 56 or engaging the wall of the second body tissue with an amount of force less than an amount force required to cause the second body tissue proximate the opening 64 therethrough from separating from the first body tissue 52.

As depicted in FIG. 6E, the handle 12 (not depicted in FIG. 6E) may be adjusted to advance the outer sheath 27 in a proximal direction relative to the inner body 28 and/or the stent 34. Advancing the outer sheath 27 in the proximal direction relative to the inner body 28 and/or the stent 34 may allow a second flange 70 of the stent 34 to expand in the first body lumen 50. Before, during, or after advancing the outer sheath 27 in the proximal direction, the endoscope 60 may be withdrawn and/or disengaged from the first body tissue 52 to allow the second flange 70 to expand at the first body tissue 52 proximate the opening 62 through the first body tissue 52.

Once the first flange 68 and the second flange 70 of the stent 34 are expanded, respectively, in the second body lumen 54 and the first body lumen 50, the first body lumen 50 may be fluidly coupled with the second body lumen 54 via the stent 34. After the stent 34 is placed, the endoscope 60 and/or the device 10 may be removed from the subject and/or one or more other suitable procedures may be initiated.

The materials that can be used for the various components of device 10 and the various elements thereof or used therewith and disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A catheter assembly, comprising:
an outer sheath having an outer sheath lumen extending therethrough;
an inner body having a proximal end, a distal end, and an inner body lumen extending therethrough, a portion of the inner body defining a stent holding region;
a distal tip positioned proximate to the distal end of the inner body; and
a flexible region between a distal end of the stent holding region and a proximal end of the distal tip.

2. The catheter assembly of claim 1, wherein the stent holding region is configured to hold a stent such that the distal end of the stent is spaced from the proximal end of the distal tip a distance in a range of 3 millimeters (mm) to 8 mm.

3. The catheter assembly of claim 1, wherein the distal tip comprises:
a tapered tip having a proximal end having a first diameter, a distal end having a second diameter, and a guidewire lumen extending therethrough, wherein the first diameter is greater than the second diameter;
a distal tip base disposed on the proximal end of the tapered tip, the guidewire lumen extending through the distal tip base; and
wherein the tapered tip has a first length and the distal tip base has a second length that is shorter than the first length.

4. The catheter assembly of claim 3, wherein the distal tip base has a length of 1 mm.

5. The catheter assembly of claim 1, wherein the distal tip is more rigid than the inner body at a portion of the inner body between the distal end of the stent holding region and the proximal end of the distal tip.

6. The catheter assembly of claim 1, wherein the inner body has a plurality of layers including an inner layer formed of a polyimide, an outer layer formed of a polyether block amide, and a middle layer formed of a braided material.

7. The catheter assembly of claim 1, wherein the inner body comprises a braided layer having a first per-inch-cross (PIC) count distal of a distal end of the stent holding region and a second PIC count proximal of a proximal end of the stent holding region, the first PIC count is greater than the second PIC count.

8. The catheter assembly of claim 1, wherein the inner body comprises a braided layer having first pattern distal of a distal end of the stent holding region and a second pattern proximal of a proximal end of the stent holding region.

9. The catheter assembly of claim 1, wherein the inner body comprises a braided layer formed from a wire having a first diameter distal of a distal end of the stent holding region and a second diameter proximal of a proximal end of the stent holding region, the second diameter is greater than the first diameter.

10. The catheter assembly of claim 1, wherein the inner body is formed from one or more polymer materials having a durometer in a range of 40 Shore D to 90 Shore D.

11. The catheter assembly of claim 1, further comprising:
a handle coupled with the inner body and the outer sheath, wherein actuation of a first portion of the handle advances the inner body in a first direction relative to the outer sheath and actuation of a second portion of the handle advances the outer sheath relative to the inner body and in a second direction opposing the first direction.

12. A catheter assembly, comprising:
an outer sheath having an outer sheath lumen extending therethrough;
an inner body having a proximal end, a distal end, and an inner sheath lumen extending therethrough, a portion of the inner body receivable within the outer sheath lumen of the outer sheath;
a distal tip positioned proximate to the distal end of the inner body, the distal tip comprising:
a tapered tip having a proximal end having a first diameter, a distal end having a second diameter, and a guidewire lumen extending therethrough, wherein the first diameter is greater than the second diameter;
a distal tip base disposed on the proximal end of the tapered tip, the guidewire lumen extending through the distal tip base; and
a conductive cutting member coupled with the tapered tip; and
a stent constrained on the inner body with a distal end of the stent spaced from a proximal end of the distal tip base.

13. The catheter assembly of claim 12, further comprising:
a stent holder coupled with the inner body and configured to couple the stent relative to the inner body with the distal end of the stent spaced from the proximal end of the distal tip base.

14. The catheter assembly of claim 12, wherein a distance between the distal end of the stent and the proximal end of the distal tip base is in a range of 3 millimeters (mm) to 8 mm.

15. The catheter assembly of claim 12, wherein the tapered tip has a first length and the distal tip base has a second length that is shorter than the first length.

16. The catheter assembly of claim 12, wherein the distal tip is more rigid than the inner body at a portion of the inner body between the distal end of the stent and the proximal end of the distal tip base.

17. The catheter assembly of claim 12, wherein the inner body is formed from one or more polymer materials having a durometer in a range of 40 Shore D to 90 Shore D.

18. A method of using a catheter assembly, the method comprising:
engaging a distal end of an endoscope having endoscopic ultrasound (EUS) visualization with a surface of or in contact body tissue defining a first body lumen;
advancing the catheter assembly through the endoscope, while a stent is constrained between an inner body and an outer sheath of the catheter assembly;
inserting a guidewire through the endoscope and into a second body lumen;
advancing an inner body and a distal tip of the catheter assembly into the second body lumen over the guidewire;
advancing the inner body over the guidewire and relative to the outer sheath in a first direction to deploy a distal end of the stent within the second body lumen; and
advancing the outer sheath relative to the inner body in a second direction to deploy a proximal end of the stent within the first body lumen.

19. The method of claim 18, wherein the distal end of the endoscope is engaged with the surface of the body tissue defining the first body lumen with the distal tip and the inner body in the second body lumen and while advancing the inner body relative to the outer sheath to deploy the distal end of the stent within the second body lumen.

20. The method of claim 18, wherein the distal end of the stent is spaced proximal of a proximal end of the distal tip while the stent is constrained between the inner body and the outer sheath.
